(19) United States
(12) Patent Application Publication
(10) Pub. No.: US 2024/0122844 A1
(43) Pub. Date: Apr. 18, 2024

(54) CARRIER FOR SUSTAINED-RELEASE OF DRUG AND USE THEREOF

(71) Applicant: KUNMING QINGCHENG HEALTHCARE TECH LTD., Kunming (CN)

(72) Inventors: Lei ZHANG, Kunming (CN); Kewang LU, Kunming (CN); Yu SUN, Kunming (CN); Tuojiang WU, Kunming (CN); Xiaojun SHI, Kunming (CN); Ling ZHU, Kunming (CN); Jie WEI, Kunming (CN); Yang SONG, Kunming (CN); DAVID LUK, Kunming (CN)

(21) Appl. No.: 18/234,230

(22) Filed: Aug. 15, 2023

(30) Foreign Application Priority Data
Oct. 12, 2022 (CN) 202211245682.3

(51) Int. Cl. A61K 9/00; A61K 9/06; A61K 47/14; A61K 47/44

(52) U.S. Cl. CPC A61K 9/0019; A61K 9/06; A61K 47/14; A61K 47/44

(57) ABSTRACT

The present disclosure relates to a carrier for sustained-release of a drug and use thereof. The carrier is used to carry a drug. The carrier is in a gel state, a semi-solid state, or a solid state. The carrier includes a polyol fatty acid ester as a main component and a hydrogenated vegetable oil dissolved in the polyol fatty acid ester. The state of the sustained-release carrier can be adjusted by adjusting the amount of the dissolved hydrogenated vegetable oil.

### CROSS-REFERENCE TO RELATED APPLICATIONS

This application is based on and claims priority to Chinese patent application No. 202211245682.3, filed on Oct. 12, 2022, the content of which is hereby incorporated by reference in its entirety.

### TECHNICAL FIELD

The present disclosure relates to the field of pharmaceutical technology, and particularly to a carrier for sustained-release of a drug or compound and use thereof.

### BACKGROUND

Periodontitis is a local bacterial infectious disease caused by pathogenic microorganisms in periodontal pockets. The destruction of periodontal tissue is the result of the interaction between complex subgingival microflora and specific host defense mechanisms. Periodontal potential pathogens are sensitive to a variety of antimicrobial agents, and there are many administration methods, including gargling, rinsing, systemic administration, and topical administration with sustained-release drugs or controlled-release drugs. The key to topical administration is that the drug must reach the lesion site and be maintained at a sufficient concentration for a sufficient time period. The drug should target the residual bacteria in periodontal pockets, soft tissue walls of pockets, exposed cementum, and root dentin. However, it is shown in experiments that many drugs for topical administration could not reach all the above areas. The minimum inhibitory concentration (MIC) and minimum bactericidal concentration (MBC) of drugs for bacteria in vitro culture is not applicable to bacteria in vivo environment. A higher drug concentration is required for antimicrobial action against bacteria in the plaque biofilm. It is necessary to maintain the effective drug concentration at the action site for a sufficient time period to cause the desired effects. Gingival crevicular fluid is the serum exudate from the gingival sulcus. The gingival sulcus has a low static volume and a high flow rate of fluid. It is estimated that in a periodontal pocket with a depth of 5 mm, the gingival sulcus fluid is displaced about 40 times per hour, so the subgingival local concentration of an antimicrobial agent will drop rapidly. The half-life of a drug in the gingival sulcus is about 1 min. The duration of action of different drugs also varies. For example, chlorhexidine requires relatively short exposure time as it kills microorganisms by destroying the cell membrane of the microorganisms. In contrast, bacteriostatic agents such as tetracycline, which inhibit bacterial protein synthesis, require a relatively longer exposure time.

Over the past 20 years, topical administration, typically in sustained-release dosage form or controlled-release dosage form that can release drugs slowly and sustainably, have become the main administration method in pharmacotherapy for periodontal diseases because of their many advantages such as reduced side effects compared with systemic administration, high local drug concentration, long duration of action, no or minimal systemic intake, etc. Drug-loaded microparticles or nanoparticles, drug-loaded films, non-filling medication, or injectable gels can be topically administered. Except for injectable gels, the other dosage forms are complicated for operation, laborious, and difficult to reach specific sites. The current matrix carriers of injectable gels are polylactic acid (PLA) and poly-L-lactic acid (PLLA), and the drugs are antibiotics, such as tetracycline, minocycline, clindamycin, ofloxacin, etc. Polylactic acid and poly-L-lactic acid are originally used for sustained release implants in the body. They have long degradation time (up to six months) and can form a rigid solid in a liquid environment. As such, they are unfavorable for periodontal tissue regeneration and repair. Antibiotics have the disadvantage of developing drug resistance, and some bacterial strains are not sensitive to tetracycline. Thus, there is a clinical need for an injectable periodontal gel composed of a flexible carrier and effective antimicrobial substances.

The commercially available periodontal sustained-release preparations mainly include the following types: 1. Tetracycline fiber (Actisite), a linear preparation, contains 25% tetracycline hydrochloride and is made of non-degradable vinyl acetate polymer as a sustained-release material. After being filled into the lesion site, the fiber is fixed in the periodontal pocket by sealing the periodontal pocket with a cyanoacrylate adhesive. Its disadvantage is that the carrier material could not be biodegradable and needs to be removed after the treatment. 2. Doxycycline gel injection (Atridox), a biodegradable in-situ gel injection, is composed of 10% doxycycline hydrochloride and 90% PLA dissolved in N-methylpyrrolidone (NMP), which are packed into two syringes respectively. When used, the two syringes are coupled, and the contents are pushed back and forth before injecting into the periodontal pocket. The polymer becomes semi-solid upon contact with gingival crevicular fluid. Its disadvantage is that sustained-release effect is poor with an initial burst effect, and the use of NMP as a solvent has yet to be verified for safety. In addition, in order to prevent the drug from flowing back into the oral cavity from the periodontal pocket, the periodontal pocket needs to be sealed with an adhesive. 3. Minocycline ointment injection (Periocline/Dentomycin) contains 2% minocycline and a carrier composed of hydroxyethyl cellulose (HEC) and other excipients. Periocline with a trade name of Paleo is commercially available in China. Its disadvantage is that sustained-release effect is poor with an initial burst effect. 4. Minocycline microsphere injection (Arestin) contains 25% minocycline, and is injected into the periodontal pocket using a periodontal syringe. Its disadvantage is that the microsphere powder is difficult to be maintained in the periodontal lesion site for a long time, which is not conducive to maintaining an effective drug concentration for a long time. 5. Metronidazole gel injection (Elyzol), a white-like semi-solid suspension, contains 40% metronidazole benzoate and a carrier composed of monooleate, vegetable oil, etc. The drug is injected into the gingival sulcus with a syringe. Its disadvantage is that the gel transforms into a high-viscosity liquid crystal state upon contacting with water in the periodontal pocket, which results in a short residence time in the periodontal pocket and is not conducive to maintaining an effective drug concentration for a long time.

The most commonly used solvents for poly(lactic-co-glycolic acid) (PLGA) or PLLA are dichloromethane and NMP. It has been confirmed by the World Health Organization that dichloromethane is a possible human carcinogen. It has been described by the US Environmental Protection Agency that NMP is a substance with low developmental toxicity. After entering the body, PLGA, PLA, or PLLA dissolved in NMP quickly forms a porous body due to the rapid solvent diffusion and dispersion. A large amount of water in the body fluid enters the polymer and releases the active substance upon contact therewith, resulting in a great volume of initial burst and a short-lasting sustained-release effect. After the solvent NMP of PLGA and PLA is released, a rigid solid is left, which is not conducive to periodontal tissue regeneration. As for other solvents, such as ethyl acetate, polyethylene glycol-400 (PEG-400) and dimethyl sulfoxide (DMSO), ethyl acetate stimulates the eyes, nose, and throat, can cause gum bleeding due to vascular and nerve disorders, and can cause eczema-like dermatitis. PEG-400 is used as a solvent for a matrix material of a sustained-release gel. After injection into the human body, PEG-400 is dissolved in water and diffused rapidly, causing a large amount of water in body fluids to enter the polymer and release the active substance upon contacting therewith, resulting in a great volume of initial burst and a short-lasting sustained-release effect. DMSO is soluble in water, and thus similar to NMP, has a great volume of initial burst and a short-lasting sustained-release effect.

### SUMMARY

A carrier with a drug sustained-release function is provided for carrying a drug to achieve a drug sustained-release effect. The carrier is in a gel state, a semi-solid state, or a solid state. The carrier includes a polyol fatty acid ester as a main component and a hydrogenated vegetable oil dissolved in the polyol fatty acid ester. The state of the sustained-release carrier can be adjusted by adjusting the amount of the dissolved hydrogenated vegetable oil.

In some embodiments, the polyol fatty acid ester can be at least one of glyceryl triacetate, caprylic/capric glycerides, glyceryl monocaprate, glyceryl monocaprylate, glyceryl dicaprylate, or glyceryl tricaprylate. In some embodiments, the polyol fatty acid ester can be glyceryl monocaprylate, glyceryl dicaprylate, or glyceryl tricaprylate. In an embodiment, the polyol fatty acid ester can be glyceryl monocaprylate (CAS No. 19670-49-6). Glyceryl monocaprylate (also named as capryl mono glyceride, CMG) is an intermediate metabolite of fat, often used as a non-toxic, high-efficiency and broad spectrum preservative, and can inhibit Gram-negative bacteria, molds, and yeasts. Like fat, glyceryl monocaprylate can be decomposed and metabolized in the body, and finally become carbon dioxide and water without any accumulation and adverse reactions. In the present disclosure, the polyol fatty acid ester is used as a solvent for dissolving the hydrogenated vegetable oil, and the state of the sustained-release carrier can be adjusted by adjusting the amount of the dissolved hydrogenated vegetable oil. For example, the sustained-release carrier can be adjusted to be in a gel state for preparing an injectable gel. By injecting the gel into the periodontal lesion site, the gel can conveniently and accurately reach the lesion without falling off since the sustained-release carrier has a flexible structure and is adhesive to the teeth and gingival tissue. The drug dissolved in the sustained-release carrier can continuous to release through diffusion, so as to achieve a stable sustained-release effect without burst release. When the drug release cycle is end, the remaining gel is gradually degraded, and the degradation products are carbon dioxide and water. Therefore, the gel does not need to be removed after the treatment and not affect the regeneration and repair of periodontal tissue.

In some embodiments, the hydrogenated vegetable oil can be at least one of hydrogenated coconut oil, hydrogenated palm oil, hydrogenated castor oil, hydrogenated tea seed oil, hydrogenated linseed oil, or hydrogenated hempseed oil. In an embodiment, the hydrogenated vegetable oil can be hydrogenated castor oil. In the present disclosure, the hydrogenated vegetable oil is contained mainly to adjust the state of the sustained-release carrier, and in addition makes the sustained-release carrier have adhesion. It has been proved by experimental research that the hydrogenated vegetable oil also can improve the sustained-release effect, and thus the use of the hydrogenated vegetable oil improves the sustained-release effect of the carrier to a certain extent.

In some embodiments, the carrier also includes one or more of a thickening

agent, an adhesive, a humectant, a preservative, a colourant, or a flavoring agent that is compatible to the other components. For example, the thickening agent can be PEG, cellulose, a long-chain fatty alcohol, or a long-chain fatty acid, with an average molecular weight greater than 1000, or an ester, a lecithin, a phytosterol, etc. thereof. The humectant can be a polyhydric alcohol, hyaluronic acid or a salt thereof, a plant polysaccharide, etc.

The preservative can be a guanidine cationic antibacterial agent, a quaternary ammonium salt, an antibiotic, etc. The colorant can be a food or drug pigment. The flavoring agent can be a sweetener or a flavor. These components are generally used as auxiliary agents, and their addition amounts are relatively small, generally around 0.01% to 10% by mass of the preparation. Alternatively, these components can be directly omitted.

In some embodiments, the mass ratio of the polyol fatty acid ester to the hydrogenated vegetable oil is (40 to 97):(2 to 40), and the mass ratio is mainly selected according to the required state of the carrier.

In some embodiments, the amount of the drug is 0.01% to 10.0% by mass in the preparation, which is determined according to the type and dosage of the drug, and for example, can be 0.01%, 0.05%, 0.1%, 0.5%, 0.8%, 0.9%, 1.0%, 1.2%, 1.6%, 1.8%, 2.0%, 2.5%, 2.8%, 4.0%, 4.2%, 4.6%, 4.8%, 5.0%, 5.5%, 6.8%, 7.5%, 8.4%, 9.6%, 10.0%, etc.

In some embodiments, the polyol fatty acid ester in the carrier is glyceryl monocaprylate, the CAS number of which is 19670-49-6.

In some embodiments, the hydrogenated vegetable oil is hydrogenated castor oil.

A pharmaceutical preparation includes the above-described carrier and a drug delivered by the carrier.

Use of the carrier with the drug sustained-release function in preparing a pharmaceutical preparation, includes adding a drug to the above-described carrier to form the pharmaceutical preparation. The pharmaceutical preparation is applied to a mucous membrane of an animal or/and human body, such as the oral mucosa, nasal mucosa, conjunctiva, vaginal mucosa, rectal mucosa, etc.

In some embodiments, the pharmaceutical preparation is an injectable gel preparation for treating an oral disease.

In some embodiments, the injectable gel preparation is mainly made of a polyol fatty acid ester, a hydrogenated vegetable oil, and a drug. In the injectable gel preparation, the amount of the polyol fatty acid ester is 40% to 97% by mass, for example, such as 40.0%, 45.0%, 57.0%, 63.5%, 75.0%, 78.0%, 80.0%, 85.2%, 88.5%, 90.0%, 92.0%, 94.0%, 97.0%, etc.; the amount of the hydrogenated vegetable oil is 2% to 40% by mass, such as 2.0%, 7.0%, 8.5%, 9.0%, 10.0%, 13.5%, 15.0%, 18.0%, 18.5%, 20.0%, 25.5%, 30.0%, 32.5%, 36.5%, 40.0%, etc.; and the balance is the drug and optional auxiliary agents. The auxiliary agents can be one or more of a thickening agent, an adhesive, a humectant, a preservative, a colourant, or a flavoring agent that is compatible to the other components.

A method for preparing an injectable gel preparation includes following steps of:
accurately weighing a predetermined amount of a polyol fatty acid ester, taking 70w % to 90w % of the predetermined amount of the polyol fatty acid ester to add with a hydrogenated vegetable oil, heating to 70° C. to 80° C. to dissolve the hydrogenated vegetable oil completely, and slowly cooling to 40° C. under stirring to obtain a first mixture;
adding a drug to the remaining 10 w % to 30 w % of the predetermined amount of the polyol fatty acid ester, and adding an auxiliary agent with the drug if any, and then homogeneously dispersing to obtain a second mixture; and
adding the second mixture to the first mixture at 40° C., stirring evenly, and cooling slowly to room temperature to obtain the preparation.

The embodiments of the present disclosure provide a carrier with a drug sustained-release function and use thereof, which is mainly used to prepare a periodontal local sustained-release preparation. The carrier mainly includes a polyol fatty acid ester and a hydrogenated vegetable oil, and then added with a drug to achieve sustained-release of the drug. The sustained-release carrier is a biodegradable flexible matrix, which is adhesive to teeth and gingival tissues and can overcome the shortcomings of traditional periodontal local sustained-release preparations.

The beneficial effects of one or more of the present embodiments of the present disclosure are as follows:
1. The sustained-release carrier of the present embodiments does not destroy or reduce the efficacy of the active substance of the drug. As the sustained-release effect is achieved by drug diffusion, the sustained release is stable and effective, avoiding the problem of burst release.
2. The sustained-release carrier of the present embodiments is a biodegradable flexible matrix, avoiding the problem of forming a rigid solid to stimulate the periodontal tissue due to the rapid release of the solvent during the sustained-release period of the drug. The degradation products of the sustained-release carrier are carbon dioxide and water, which are non-toxic and harmless and will not affect the regeneration and repair of the periodontal tissue. Thus, the sustained-release carrier does not need to be removed after treatment.
3. The solvent in the sustained-release carrier of the present embodiments is safe, biodegradable, biocompatible, and water-insoluble. The solvent combined with a hydrogenated vegetable oil can be prepared into various states of the sustained-release carrier, and is adhesive. The carrier can be used as a periodontal injectable gel to easily and accurately adhere to the teeth and gingival tissue, thereby better exerting local therapeutic effects. Therefore, the carrier has obvious technical advantages and great market potential as compared with current injectable gels.

### BRIEF DESCRIPTION OF THE DRAWINGS

FIG. 1 shows absorbance spectra of chlorhexidine acetate released in purified water for 1 to 15 days from a carrier of group B (formulation 3).

FIG. 2 shows absorbance spectra of chlorhexidine acetate released in purified water for 13 to 27 days from a carrier of group B (formulation 3).

FIG. 3 shows absorbance spectra of chlorhexidine acetate released in simulated body fluid for 1 to 15 days from a carrier of group C (formulation 2).

FIG. 4 shows absorbance spectra of chlorhexidine acetate released in artificial saliva for 1 to 15 days (respectively on day 1, 3, 5, 7, 9, 11, 13, 15) from the carrier of group D (formulation 4).

FIG. 5 shows absorbance spectra of metformin hydrochloride in purified water for 1-5 days from the carrier of group F (formulation 6).

FIG. 6 shows absorbance spectra of metformin hydrochloride in purified water for 1-5 days from the carrier of group G (formulation 5).

FIG. 7 shows absorbance spectra of ranitidine hydrochloride in purified water for 1-6 days from the carrier of group H (formulation 7).

FIG. 8 shows absorbance spectra of vitamin B1 in purified water for 1-6 days from the carrier of group I (formulation 8).

FIG. 9 shows absorbanceg spectra of benzalkonium chloridein purified water for 1-6 days from the carrier of group J (formulation 9).

### DETAILED DESCRIPTION

The present disclosure will be described in detail below with reference to the accompanying drawings.

In order to make the above objectives, features and advantages of the present disclosure more clear and understandable, the present disclosure will be described in detail. It should be understood that the exemplary examples are merely for the purpose of understanding of the present disclosure but are not meant to limit the scope thereof.

Release of different drugs by the sustained-release carrier.

### I. SUSTAINED-RELEASE EFFECT ON CHLORHEXIDINE ACETATE

### 1. Experimental Materials

1) Test samples: formulations 1 to 4;
2) Drug release medium: purified water (Wahaha®), artificial saliva (from Chuangfeng Technology Co., Ltd.), and simulated body fluid (from Phygene Life Sciences Co., Ltd.).

### 2. Experimental Equipment

1) UV-visible spectrophotometer (SP-756PC).

### 3. Group Setting

1) group A where the sample is 0.1946 g of formulation 2 and the medium is 30.0 g of purified water;
2) group B where the sample is 0.2045 g of formulation 3 and the medium is 30.11 g of purified water;
3) group C where the sample is 0.1983 g of formulation 2 and the medium is 30.06 g of simulated body fluid;
4) group D where the sample is 0.2022 g of formulation 4 and the medium is 30.06 g of artificial saliva;
5) group E where the sample is 0.2047 g of formulation 1 and the medium is 30.07 g of artificial saliva.

### 4. Experimental Method

1) The test samples from different formulations were respectively introduced into 2 mL-syringes. 2) About 0.2 g of the test samples were squeezed out from the syringes without a needle into 30 mL transparent bottles respectively. The test samples in the bottles were accurately weighed. 3) 30.0 g of the drug release medium was added to each bottle. 4) The absorbance spectrum of the liquid in each bottle was initially scanned and the absorbance was measured at the wavelength of 254 nm at 24th hour, and then scanned and measured every 48 hours. 5) The purified water (Wahaha®), artificial saliva (from Chuangfeng Technology Co., Ltd.), simulated body fluid (from Phygene Life Sciences Co., Ltd.) were used as the reference solutions. The liquids to be tested were taken from the bottles with the carriers and put back to the bottles after the spectral scanning.

### 5. Experimental Results

TABLE 1
Drug release in water from sustained-release carriers

| Time | Absorbance (A) of each group | | | | |
|---|---|---|---|---|---|
| (days) | Group A | Group B | Group C | Group D | Group E |
| 1 | 0.113 | 0.126 | 0.064 | 0.086 | 0.070 |
| 3 | 0.361 | 0.328 | 0.104 | 0.208 | 0.255 |
| 5 | 0.543 | 0.504 | 0.143 | 0.396 | 0.422 |
| 7 | 0.644 | 0.660 | 0.172 | 0.536 | 0.565 |
| 9 | 0.698 | 0.742 | 0.198 | 0.701 | 0.710 |
| 11 | 0.716 | 0.810 | 0.208 | 0.845 | 0.829 |
| 13 | 0.743 | 0.846 | 0.209 | 1.020 | 0.909 |
| 15 | 0.744 | 0.865 | 0.208 | 1.183 | 0.962 |
| 17 | 0.746 | 0.882 | 0.204 | 1.281 | 1.030 |
| 19 | — | 0.895 | 0.204 | 1.371 | 1.075 |
| 21 | — | 0.906 | — | 1.430 | 1.144 |
| 23 | — | 0.911 | — | A great | 1.140 |
| | | | | amount of | |
| | | | | precipitation | |
| | | | | produced | |
| 25 | — | 0.918 | — | — | — |
| 27 | — | 0.924 | — | — | — |

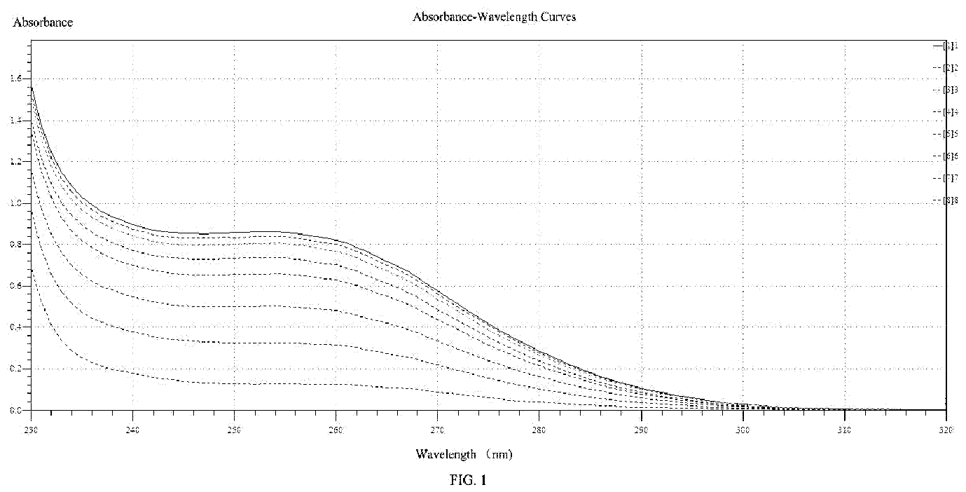

FIG. 1 shows the absorbance spectra of chlorhexidine acetate released in purified water for 1 to 15 days (respectively on day 1, 3, 5, 7, 9, 11, 13, 15) from the carrier of group B (formulation 3). It can be seen from FIG. 1 that the maximum absorption peak is at the wavelength of 254 nm, and the released amount uniformly and regularly decreases with time.

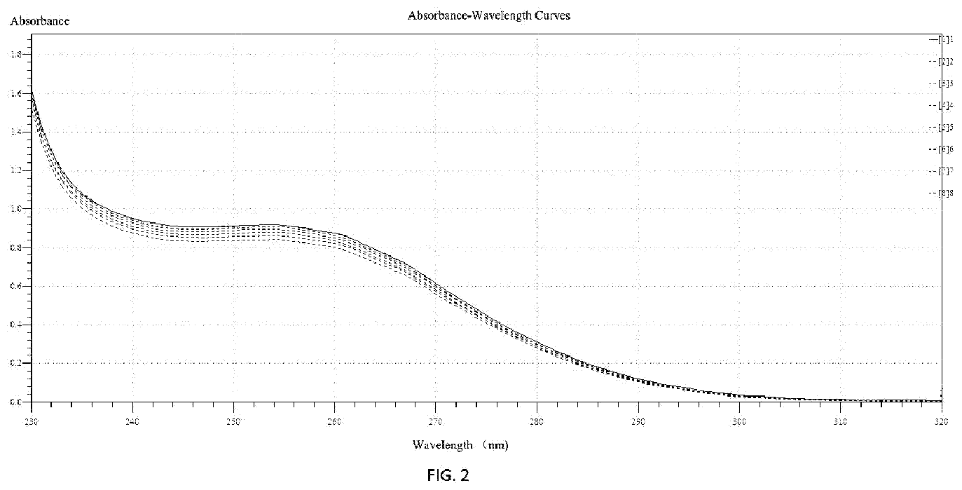

FIG. 2 shows the absorbance spectra of chlorhexidine acetate released in purified water for 13 to 27 days (respectively on day 17, 19, 21, 23, 25, 27) from the carrier of group B (formulation 3). Combined with FIG. 1, it can be seen that the release is completed in about 15 days.

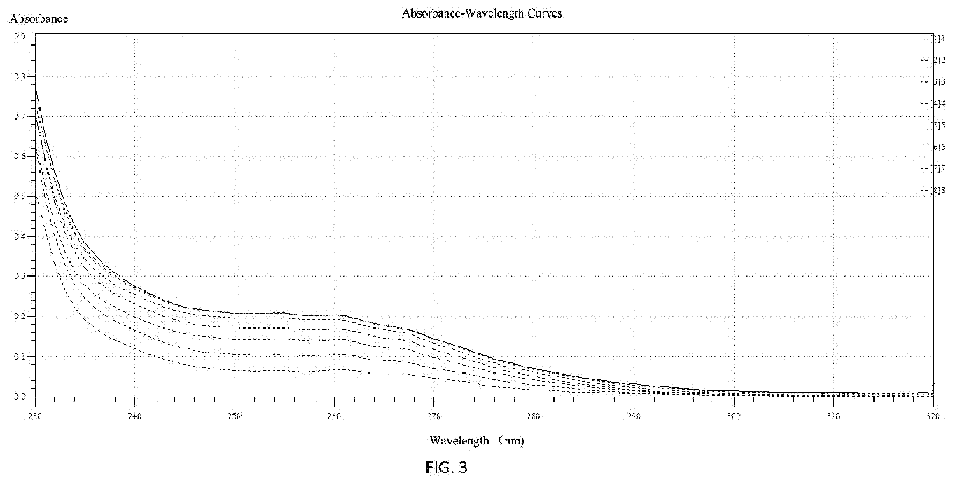

FIG. 3 shows the absorbance spectra of chlorhexidine acetate released in

simulated body fluid for 1 to 15 days (respectively on day 1, 3, 5, 7, 9, 11, 13, 15) from the carrier of group C (formulation 2). It can be seen from FIG. 3 that the maximum absorption peak is at the wavelength of 254 nm, and the released amount uniformly and regularly decreases with time.

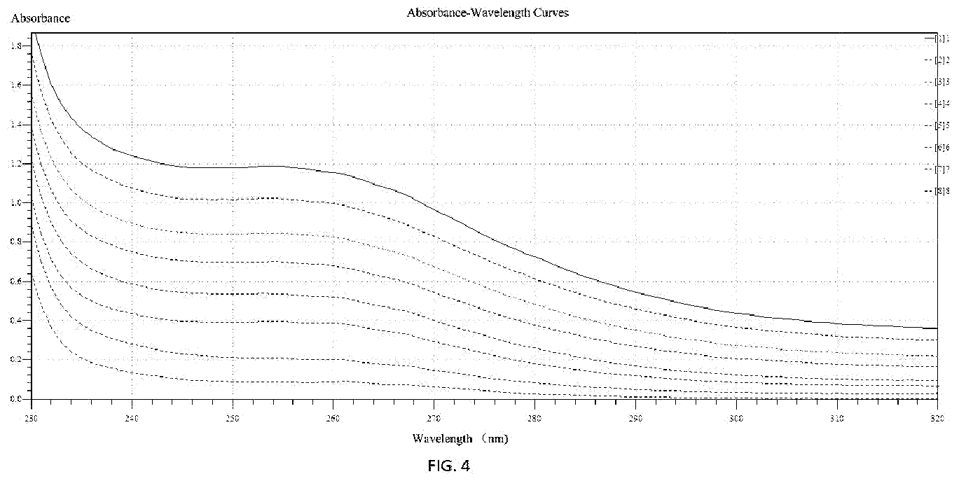

FIG. 4 shows the absorbance spectra of chlorhexidine acetate released in artificial saliva for 1 to 15 days (respectively on day 1, 3, 5, 7, 9, 11, 13, 15) from the carrier of group D (formulation 4). It can be seen from FIG. 4 that the maximum absorption peak is at the wavelength of 254 nm, and the released amount uniformly and regularly decreases with time.

### 6. Experimental Conclusion

When the absorbance (A) is increased less than 0.01, it is considered that the drug has been substantially released. From the absorbance values of groups A and B, it can be concluded that the sustained-release carriers can uniformly and regularly release chlorhexidine acetate in purified water, and the release can last about 15 days.

From the absorbance values of group C, it can be concluded that due to the complex composition of the simulated body fluid, the measured absorbance is obviously affected by the reference solution. However, it can still be observed that the amount of the drug released from the sustained-release carrier uniformly and regularly decreases with time in the simulated body fluid. From the data it can be seen that the release can substantially last about 9 days. However, due to the fact that the content of chlorhexidine acetate is 0.5%, considering the influence of the reference solution and upon a comprehensive data analysis of groups A to E in Table 1, it can be inferred that the release should also last about 9 to 15 days.

From the absorbance values of groups D and E, it can be concluded that due to the complex composition of the artificial saliva, the measured absorbance is obviously affected by the reference solution. However, it can still be observed that the amount of the drug released from the sustained-release carrier uniformly and regularly decreases with time in the artificial saliva. From the data it can be seen that the release can substantially last more than 15 days. However, due to the fact that the content of chlorhexidine acetate is 0.5%, considering the influence of the reference solution and upon a comprehensive data analysis of groups A to E in Table 1, it can be inferred that the release should also last about 15 days.

### II. SUSTAINED-RELEASE EFFECTS ON METFORMIN HYDROCHLORIDE, RANITIDINE HYDROCHLORIDE, VITAMIN B1, AND BENZALKONIUM CHLORIDE

### 1. Experimental Materials

1) Test samples: formulation 5, formulation 6, formulation 7, formulation 8, formulation 9;
2) Drug release medium: purified water (Wahaha®).

### 2. Experimental Equipment

1) UV-visible spectrophotometer (SP-756PC).

### 3. Group Setting

1) group F where the sample is 0.1990 g of formulation 6 and the medium is 30.11 g of purified water;
2) group G where the sample is 0.2016 g of formulation 5 and the medium is 30.14 g of purified water;
3) group H where the sample is 0.2087 g of formulation 7 and the medium is 30.11 g of purified water;
4) group I where the sample is 0.2016 g of formulation 8 and the medium is 30.12 g of purified water;
5) group J where the sample is 0.0975 g of formulation 9 and the medium is 30.18 g of purified water.

### 4. Experimental Method

1) The test samples with different formulations were respectively introduced into 2 mL-syringes. 2) About 0.2 g of the test samples, but for benzalkonium chloride about 0.1 g of the test sample, were squeezed out from the syringes without a needle into 30 mL transparent bottles respectively. The tests samples in the bottles were accurately weighed. 3) 30.0 g of the drug release medium was added to each bottle. 4) The absorbance spectrum of the liquid in each bottle was scanned and the absorbance at the wavelength of 232 nm for metformin hydrochloride, 313 nm for ranitidine hydrochloride, 261 nm for vitamin B1, or 219 nm for benzalkonium chloride was measured every 24 hours. 5) In groups H, I and J, the purified water (Wahaha®) was used as the reference solution. In groups F and G, blank carriers in formulations 2 and 3 without metformin hydrochloride were used to prepare the reference solutions according to the above steps 1) to 3) of “4. Experimental method”. The liquids to be tested and the reference solutions of groups F and G were taken from the bottles with the carriers and put back to the bottles after the spectral scanning.

### 5. Experimental Results

TABLE 2
Release of different amounts of drugs in
water from sustained-release carriers

| Time | Absorbance (A) of each group | | | | |
|---|---|---|---|---|---|
| (days) | Group F | Group G | Group H | Group I | Group J |
| 1 | 0.522 | 0.265 | 0.443 | 0.603 | 0.758 |
| 2 | 0.652 | 0.300 | 0.561 | 0.719 | 0.888 |
| 3 | 0.706 | 0.322 | 0.607 | 0.752 | 0.918 |
| 4 | 0.712 | 0.331 | 0.627 | 0.770 | 0.933 |
| 5 | 0.718 | 0.333 | 0.630 | 0.782 | 0.938 |
| 6 | — | — | 0.632 | 0.785 | 0.940 |

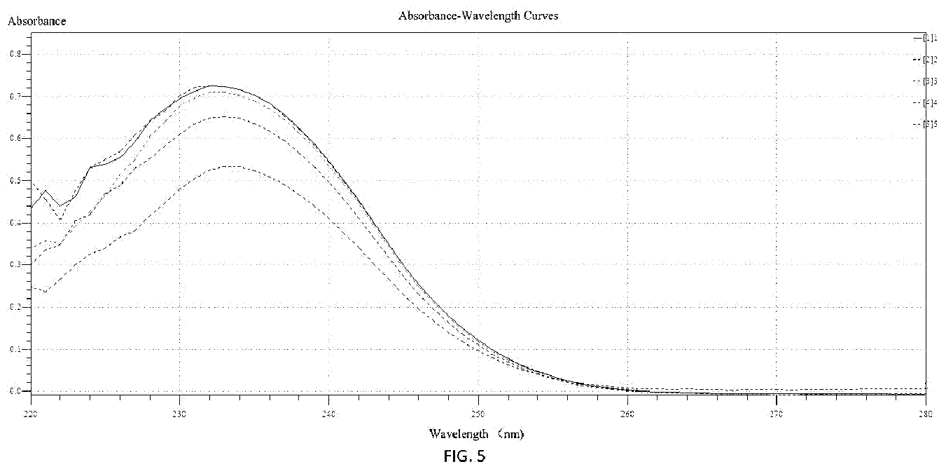

FIG. 5 shows the absorbance spectra of metformin hydrochloride released in purified water for 1 to 5 days from the carrier of group F (formulation 6). It can be seen from FIG. 5 that the maximum absorption peak is at the wavelength of 232 nm, and the released amount is decreased uniformly and regularly with time.

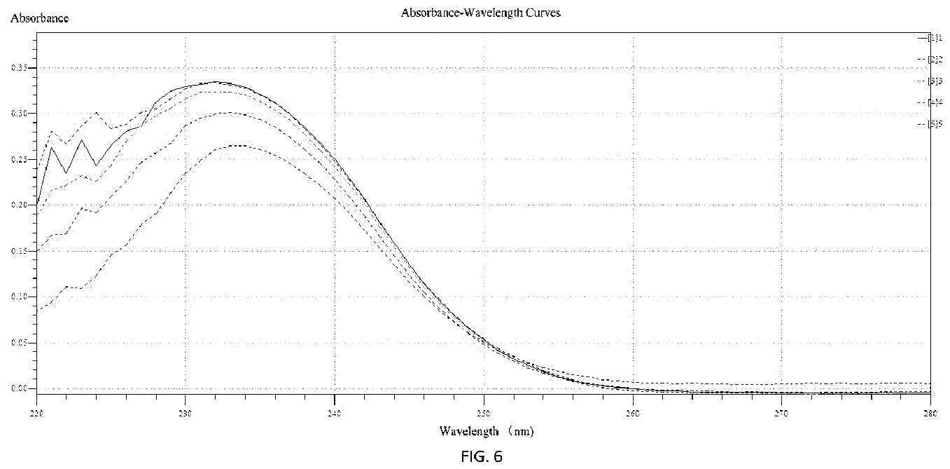

FIG. 6 shows the absorbance spectra of metformin hydrochloride released in purified water for 1 to 5 days from the carrier of group G (formulation 5). It can be seen from FIG. 6 that the maximum absorption peak is at the wavelength of 232 nm, and the released amount is decreased uniformly and regularly with time.

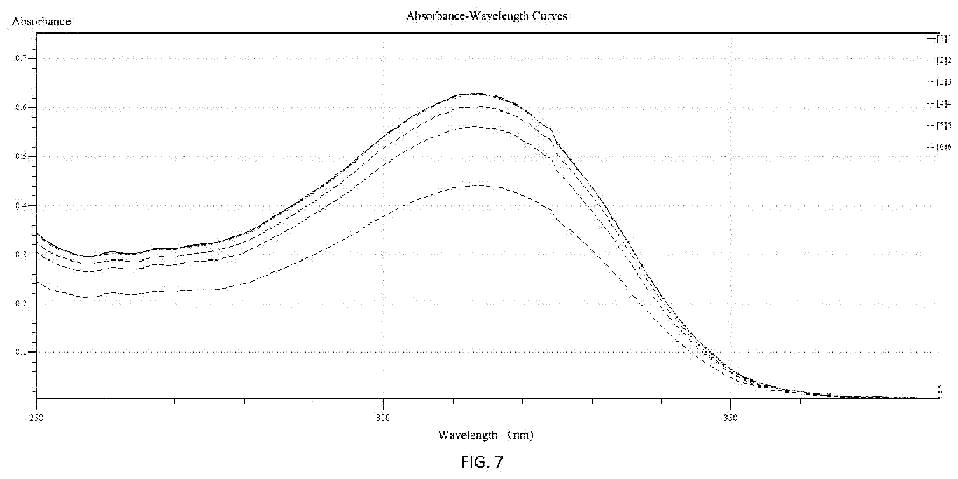

FIG. 7 shows the absorbance spectra of ranitidine hydrochloride released in purified water for 1 to 6 days from the carrier of group H (formulation 7). It can be seen from FIG. 7 that the maximum absorption peak is at the wavelength of 313 nm, and the released amount is decreased uniformly and regularly with time.

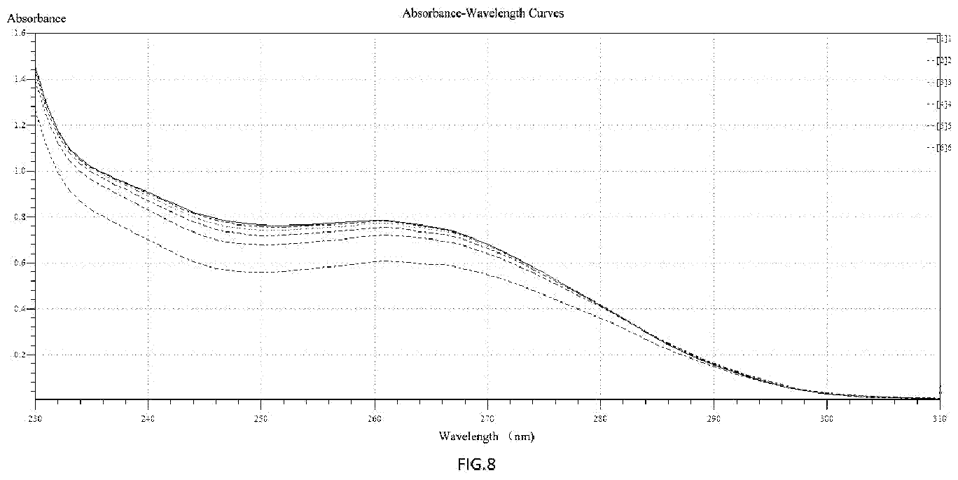

FIG. 8 shows the absorbance spectra of vitamin B1 released in purified water for 1 to 6 days from the carrier of group I (formulation 8). It can be seen from FIG. 8 that the maximum absorption peak is at the wavelength of 261 nm, and the released amount is decreased uniformly and regularly with time.

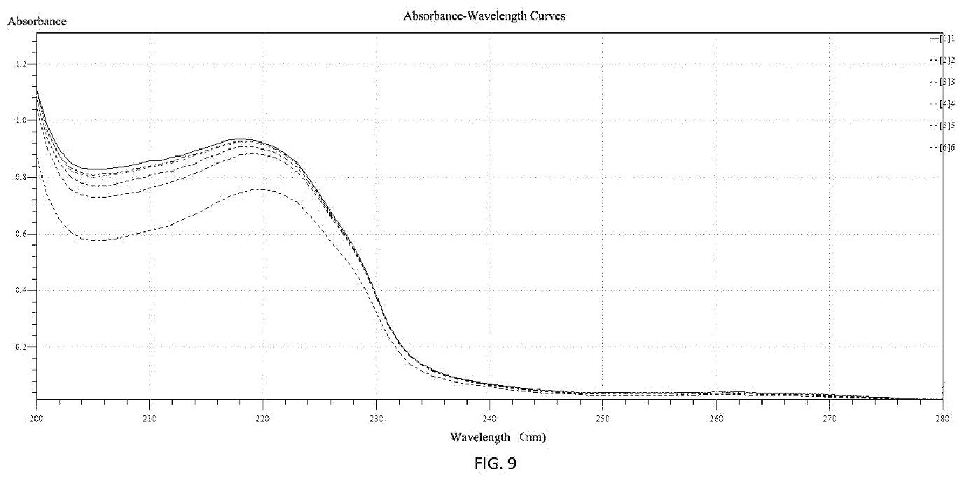

FIG. 9 shows the absorbance spectra of benzalkonium chloride released in purified water for 1 to 6 days from the carrier of group J (formulation 9). It can be seen from FIG. 9 that the maximum absorption peak is at the wavelength of 219 nm, and the released amount is decreased uniformly and regularly with time.

### 6. Experimental Conclusion

When the absorbance (A) is increased less than 0.01, it is considered that the drug has been substantially released. From the absorbance values of groups F and J, it can be concluded that the sustained-release carriers can sustainably release metformin hydrochloride in purified water, and the release can last about 3 days, and that the sustained-release carriers can sustainably release ranitidine hydrochloride, vitamin B1, benzalkonium chloride in purified water, and the release can last about 4 days.

### III. SPECIFIC EMBODIMENTS

### Example 1

The pharmaceutical preparations with different formulations were prepared using following raw materials: 40.0% to 97.0% by mass of glyceryl monocaprylate, 2.0% to 40.0% by mass of hydrogenated castor oil, and 0.01% to 10.0% by mass of chlorhexidine acetate.

The preparation method includes following steps of:
accurately weighing a predetermined amount of glyceryl monocaprylate, taking 80w % of the predetermined amount of the glyceryl monocaprylate to add with a hydrogenated castor oil, heating to 70° C. to 80° C. to dissolve the hydrogenated castor oil completely, and slowly cooling to 40° C. under stirring to obtain a first mixture;
adding chlorhexidine acetate to the remaining 20 w % of the predetermined amount of the glyceryl monocaprylate, and then homogeneously dispersing to obtain a second mixture; and
adding the second mixture to the first mixture at 40° C., stirring evenly, and cooling slowly to room temperature to obtain the preparation.

### Example 2

Table 3 shows the raw materials and their mass percentages of fourteen formulations. For each formulation, the raw materials were taken according to the mass percentages and formed into a homogeneous preparation according to the method of Example 1.

TABLE 3
Raw materials and their mass percentages
(%) in different formulations

| Formulation | HCO | GC | CHX | MH | RH | VB1 | BYZGR |
|---|---|---|---|---|---|---|---|
| 1 | 4.0 | 95.50 | 0.5 | — | — | — | — |
| 2 | 6.0 | 93.50 | 0.5 | — | — | — | — |
| 3 | 8.0 | 91.50 | 0.5 | — | — | — | — |
| 4 | 10.0 | 89.50 | 0.5 | — | — | — | — |
| 5 | 6.0 | 93.93 | — | 0.07 | — | — | — |
| 6 | 8.0 | 91.85 | — | 0.15 | — | — | — |
| 7 | 6.0 | 93.75 | — | — | 0.25 | — | — |
| 8 | 8.0 | 91.50 | — | — | — | 0.5 | — |
| 9 | 12.0 | 87.30 | — | — | — | — | 0.7 |
| 10 | 18.0 | 71.00 | 1.0 | — | — | — | — |
| 11 | 25.0 | 74.00 | — | 1.0 | — | — | — |
| 12 | 30.0 | 68.50 | — | — | 1.5 | — | — |
| 13 | 35.0 | 63.00 | — | — | — | 2.0 | — |
| 14 | 40.0 | 59.00 | — | — | — | — | 1.0 |

Note:
HCO represents hydrogenated castor oil; GC represents glyceryl monocaprylate; CHX represents chlorhexidine acetate; MH represents metformin hydrochloride; RH represents ranitidine hydrochloride; VB1 represents vitamin B1; and BZK represents benzalkonium chloride.

### Example 3

Table 4 shows the raw materials and their mass percentages of eight formulations including auxiliary agents. For each formulation, the raw materials were taken according to the mass percentages and formed into a homogeneous paste preparation according to the method of Example 1.

TABLE 4
Raw materials and their mass percentages (%) in
different formulations with auxiliary agents

| Formulation | HCO | GC | CHX | M H | Thickening agent | Humectant | Colourant |
|---|---|---|---|---|---|---|---|
| 15 | 4.0 | 93.50 | 0.5 | — | 2.0 | — | — |
| 16 | 4.0 | 94.50 | 0.5 | — | — | 1.0 | — |
| 17 | 4.0 | 95.49 | 0.5 | — | — | — | 0.01 |
| 18 | 4.0 | 94.49 | 0.5 | — | — | 1.0 | 0.01 |
| 19 | 8.0 | 90.85 | — | 0.15 | 1.0 | — | — |
| 20 | 8.0 | 90.85 | — | 0.15 | — | 1.0 | — |
| 21 | 8.0 | 91.84 | — | 0.15 | — | — | 0.01 |
| 22 | 8.0 | 91.34 | — | 0.15 | — | 0.5 | 0.01 |

Note:
HCO represents hydrogenated castor oil; GC represents glyceryl monocaprylate; CHX represents chlorhexidine acetate; MH represents metformin hydrochloride; the thickening agent is PEG-1000; the humectant is glycerol; and the colorant is a food grade blue pigment.

### Comparative Example 1

The formulation of Comparative Example 1 is: 8.0% of hydrogenated castor oil, 0.5% of chlorhexidine acetate, and 91.50% of NMP, and the preparation method is the same as that of Example 1.

### Comparative Example 2

The formulation of Comparative Example 2 is: 8.0% of hydrogenated castor oil, 0.5% of chlorhexidine acetate, and 91.50% of ethyl acetate, and the preparation method is the same as that of Example 1 except that the heating is only to 50° C.

### Comparative Example 3

The formulation of Comparative Example 1 is: 8.0% of hydrogenated castor oil, 0.5% of chlorhexidine acetate, and 91.50% of DMSO, and the preparation method is the same as that of Example 1.

### Experimental Results of Comparative Examples

Comparative Example 1: Hydrogenated castor oil can dissolve in NMP upon heating, but cannot form a homogeneous gel after cooling to room temperature.

Comparative Example 2: Hydrogenated castor oil cannot be dissolved in ethyl acetate when heated to about 50° C.

Comparative Example 3: Hydrogenated castor oil is insoluble in DMSO upon heating.

The above descriptions are only embodiments of the present disclosure, and are not intended to limit the present disclosure. Any modifications, equivalent replacements and improvements made within the spirit and principles of the present disclosure should be included in the protection scope of the present disclosure.

What is claimed is:

1. A carrier for sustained-release of a drug, wherein the carrier is in a gel state, a semi-solid state, or a solid state, the carrier comprises a polyol fatty acid ester as a main component and a hydrogenated vegetable oil dissolved in the polyol fatty acid ester.

2. The carrier of claim 1, further comprising one or more of a thickening agent, an adhesive, a humectant, a preservative, a colourant, or a flavoring agent that is compatible in the carrier.

3. The carrier of claim 1, wherein a mass ratio of the polyol fatty acid ester to the hydrogenated vegetable oil is (40 to 97):(2 to 40).

4. The carrier of claim 1, wherein the polyol fatty acid ester is selected from the group consisting of glyceryl triacetate, caprylic/capric glycerides, glyceryl monocaprate, glyceryl monocaprylate, glyceryl dicaprylate, glyceryl tricaprylate, and any combinations thereof.

5. The carrier of claim 1, wherein the polyol fatty acid ester is glyceryl monocaprylate.

6. The carrier of claim 1, wherein the hydrogenated vegetable oil is selected from the group consisting of hydrogenated coconut oil, hydrogenated palm oil, hydrogenated castor oil, hydrogenated tea seed oil, hydrogenated linseed oil, hydrogenated hempseed oil, and any combinations thereof.

7. The carrier of claim 1, wherein the hydrogenated vegetable oil is hydrogenated castor oil.

8. A pharmaceutical preparation comprising the carrier of claim 1 and a drug carried by the carrier.

9. The pharmaceutical preparation of claim 8, wherein an amount of the drug is 0.01% to 10.0% by mass in the pharmaceutical preparation.

10. The pharmaceutical preparation of claim 8 being an injectable gel preparation capable of treating an oral disease.

11. The pharmaceutical preparation of claim 10, wherein the injectable gel preparation is mainly made of the polyol fatty acid ester, the hydrogenated vegetable oil, and the drug, and in the injectable gel preparation, an amount of the polyol fatty acid ester is 40% to 97% by mass, an amount of the hydrogenated vegetable oil is 2% to 40% by mass, and a balance comprises the drug.

12. The pharmaceutical preparation of claim 11, wherein the balance further comprises an auxiliary agent, and the auxiliary agents is one or more of a thickening agent, an adhesive, a humectant, a preservative, a colourant, or a flavoring agent that is compatible in the carrier.

13. A method for preparing an injectable gel preparation comprising the carrier of claim 1, the method comprising following steps:
accurately weighing a predetermined amount of a polyol fatty acid ester, taking 70 w % to 90 w % of the predetermined amount of the polyol fatty acid ester to add with a hydrogenated vegetable oil, heating to 70° C. to 80° C. to dissolve the hydrogenated vegetable oil completely, and slowly cooling to 40° C. under stirring to obtain a first mixture;
adding a drug to the remaining 10 w % to 30 w % of the predetermined amount of the polyol fatty acid ester, and adding an auxiliary agent if any with the drug, and then homogeneously dispersing to obtain a second mixture; and
adding the second mixture to the first mixture at 40° C., stirring evenly, and cooling slowly to room temperature to obtain the preparation.

14. A method, comprising contacting the pharmaceutical preparation of claim 8 with a mucous membrane of an animal or/and human body.

15. The method of claim 14, wherein the mucous membrane is the oral mucosa, the nasal mucosa, the conjunctiva, the vaginal mucosa, or the rectal mucosa.
